(19) United States
(12) Patent Application Publication
(10) Pub. No.: US 2010/0227826 A1
(43) Pub. Date: Sep. 9, 2010

(54) Stable Flavonoid Solutions

(76) Inventor: Michel F. Mercier, Mountainside, NJ (US)

Correspondence Address: Louis C. Paul;Louis C. Paul & Associates, PLLC, 150 East 58th Street, 34th Floor, New York, NY, 10155, US

(21) Appl. No.: 12/086,248

(22) PCT Filed: Dec. 6, 2006
(86) PCT No.: PCT/US2006/046626

§ 371 Date: Jun. 9, 2008

Related U.S. Application Data
(60) Provisional application No. 60/742,902, filed on Dec. 7, 2005.

(51) Int. Cl. A61K 31/7048; A61P 17/14; A61P 29/00; A61Q 19/08; A61K 31/352; A61K 8/60; A61K 8/49

(52) U.S. Cl. 514/27; 514/456

(57) ABSTRACT

A stable flavonoid solution comprising (i) at least one flavonoid or flavonoid derivative selected from the group consisting of luteolin, myricetin, baicalin, baicalein, silymarin and kushenol, (ii) a mixture of alkyl glycols of two to seven carbon atoms, (iii) a polyol ether and (iv) an antioxidant.

### FIELD OF INVENTION

The present invention relates to stable flavonoid solutions and the use of these solutions in treating dermatologic conditions.

### BACKGROUND

From birth, and the first exposure to ultraviolet radiation (“UVR”), the skin undergoes photodamage. In time, this damage presents as hyperpigmentation of localized areas of the skin. Freckles, for example, are common pigmented childhood lesions that appear in the summer (during increased periods of UVR exposure) and fade in the fall and winter. Melasma—sometimes referred to as the “mask of pregnancy”—is another hyperpigmented condition, with blotchy areas on the cheeks, temples and forehead. This condition is often more pronounced after exposure to sunlight. Similar types of brown or ashen patches also have been observed in women using reproductive hormones (e.g., birth control pills or estrogen replacement therapy). With age, another hyperpigmented condition, solar lentigines—so-called “age” or “liver” spots—becomes more prevalent.

Hyperpigmented conditions result from increased production of melanin (a brown, UVR-absorbing pigment) by melanocytes. Melanocytes are dendritic cells present in the basal layer of the epidermis. Histologically, when stained with Fontana Masson (Silver), melanin appears black.

Melanogenesis, the synthesis of melanin, is controlled, in part, by the enzyme tyrosinase. More particularly, tyrosinase is synthesized by the ribosomes of the endoplasmic reticulum. It is then incorporated into vesicles in the Golgi apparatus, and finally released in membrane-bound vesicles within the melanocyte. Structural proteins fuse with the tyrosinase vesicles, forming melanosomes. Within the melanosomes, tyrosinase catalyzes the conversion of the amino acid tyrosine to dihydroxyphenylalanine (DOPA). (UVR can also initiate the formation of DOPA from tyrosine.) Melanosomes are released from the dendritic tips of the melanocytes and captured via phagocytosis by neighboring keratinocytes, producing skin pigmentation.

In general, there are three mechanisms by which skin pigmentation can be lightened: (i) downregulating the production of tyrosinase; (ii) inhibiting tyrosinase activity; and/or (iii) blocking uptake of melanin by the keratinocytes. See, e.g., Seiberg et al., J. Invest. Dermatol. 115:162-167 (2000). In many cases, hyperpigmentation is superficial, and responsive to topical treatments. Pigmentation deeper in the dermis may require chemical peels and/or laser treatment.

Topical skin-lightening treatments are well-known in the art and include: hydroquinone, either alone or in combination with tretinoin; azelaic acid; kojic acid, alone or in combination with glycolic acid or hydroquinone. Am. J. Clin. Derm., pp 261-268 (September-October 2000). Hydroquinone and kojic acid have been reported to be unstable in cosmetic formulations. Additionally, citing safety concerns, many European countries have banned hydroquinone as a skin-lightener.

Beyond pharmacotherapeutics, topical skin-lightening treatments based on extracts of natural products are known in the art. Arbutin, for example, is a hydroquinone glucoside isolated from the leaves of bearberry, blueberry, cowberry, cranberry and pears. The use of arbutin, alone and in combination with kojic acid, is described in U.S. Pat. Nos. 6,365,137, 6,488,917, 6,306,376 and 6,280,754.

The biochemical and physiological responses to unprotected exposure to UVR are well-characterized in the literature. After UVR exposure, cytokines (e.g., interleukins, tumor necrosis factors and prostaglandins) are released, producing erythema, edema and sometimes, pain and blistering (“sunburn”). An increase in melanin pigmentation is typically observed several days after UVR exposure. See, e.g., Farr, P. and Diffey, B., Br. J. Dermatol., 113:65-76 (1985). The correlation between UVR-induced erythema and increased pigmentation, as well as reduction in pigmentation after application of topical anti-inflammatory agents, has been reported in the literature. Takiwaki, H. et al., J. Invest Dermatol. 03 (5): 642-646 (1994).

Production of melanin may also be regulated in a paracrine manner based on indirect cell-cell interactions between melanocytes, keratinocytes and fibroblasts. For example, a relationship between melanogenesis and cutaneous inflammation has been reported in the scientific literature. More particularly, Interleukin-1α, Interleukin-6, Tumor Necrosis Factor-α have been reported to act as paracrine inhibitors of human melanocyte proliferation and melanogenesis. Swope V. B., et al., J. Invest. Dermatol. 96:180-185 (1991).

The anti-inflammatory properties of flavonoids are well-known in the art; so too, is the poor solubility of flavonoids in cosmetically-acceptable solutions. Thus, there remains a long-felt, but as yet unmet, need for cosmetically-acceptable, stable flavonoid solutions. This need is met by the present invention.

### SUMMARY OF THE INVENTION

One aspect of the present invention relates to stable flavonoid solutions comprising (i) at least one flavonoid or flavonoid derivative, (ii) a mixture of alkyl glycols of two to seven carbon atoms, (iii) a polyol ether and (iv) an antioxidant. Another aspect of the invention relates to the use of stable flavonoid solutions in treating dermatologic conditions. In one embodiment, the at least one flavonoid or flavonoid derivative is selected from the group consisting of luteolin, myricetin, baicalin, baicalein, silymarin and kushenol. In preferred and more preferred embodiments, the stable flavonoid solution comprises two and three of the above-listed flavonoids or flavonoid derivatives.

### DETAILED DESCRIPTION OF THE INVENTION

One aspect of the present invention relates to stable flavonoid solutions comprising (i) at least one flavonoid or flavonoid derivative, (ii) a mixture of alkyl glycols of two to seven carbon atoms, (iii) a polyol ether and (iv) an antioxidant.

For purposes of the present invention, a stable flavonoid solution is one that meets the following criteria: (i) After storage at 15° C. or at 45° C. for eight weeks, the solution does not have visible precipitate. (ii) After storage at 45° C. for eight weeks, the solution has an absolute change from its initial Gardner Color Scale number of less than about 1. (iii) After storage at 25° C. for eight weeks in ambient light, the solution has an absolute change from its initial Gardner Color Scale number of less than about 1. (iv) After storage at 45° C. for eight weeks, the absorbance at 520 nm of the solution changes by less than about 5% from its initial absorbance. The Gardner Color Scale is a measure of chromaticity from light yellow to red and is well-known to persons of ordinary skill in the art. More particularly, chromaticity is expressed on a scale of from 1 (lightest) to 18 (darkest). This method is further described in ASTM D1544 “Standard Test Method for Color of Transparent Liquids (Gardner Color Scale)”, which is incorporated by reference.

One aspect of the present invention relates to the use of stable flavonoid solutions of the present invention in topically-applied products for treating dermatologic conditions, including but not limited to, reducing inflammation, helping to reduce the signs and appearance of biological aging and photoaging, modulating immune function, inhibiting or slowing hair loss, and maintaining or increasing hair growth. A non-limiting example of such uses is in topically-applied skin-lightening compositions. Other dermatologic conditions which can be treated with the stable flavonoid solutions of the present invention include those described in Freedberg et al., Fitzpatrick's Dermatology in General Medicine (6th Edition, 2003),

Flavonoids

In one embodiment, the at least one flavonoid or flavonoid derivative is selected from the group consisting of luteolin, myricetin, baicalin, baicalein, silymarin and kushenol.

In another embodiment, the stable flavonoid solution comprises two of luteolin, myricetin, baicalin, baicalein, silymarin and kushenol. In one embodiment, the stable flavonoid solution comprises luteolin and one of myricetin, baicalin and baicalein. In another embodiment, the stable flavonoid solution comprises myricetin and one of luteolin, baicalin and baicalein. In a further embodiment, the stable flavonoid solution comprises baicalin and one of luteolin, myricetin and baicalein. In a still further embodiment, the stable flavonoid solution comprises baicalein and one of luteolin, myricetin and baicalin.

In a further embodiment, the stable flavonoid solution comprises three of luteolin, myricetin, baicalin, baicalein, silymarin and kushenol. A blend of baicalin, luteolin and myricetin is commercially available under the tradename Phytocytol™ BLM from M.M.P., Inc. (South Plainfield, N.J.).

Solvent System

The stable flavonoid solution of the present invention is comprised of a solvent system comprised of (I) a mixture of alkyl glycols having two to seven carbons atoms (ii) a polyol ether and (iii) an antioxidant. Preferably, the mixture of alkyl glycols having three to six carbons atoms.

The ratio of the alkyl glycol mixture to polyol ether is from about 3:2 to about 2:3. In a preferred embodiment, the ratio of the alkyl glycol mixture to polyol ether is about 1:1.

In one embodiment, at least one of the alkyl glycols is a four-carbon atom chain. In a preferred embodiment, the mixture of alkyl glycols comprises methylpropanediol, butylene diol and pentylene glycol.

A polyol ether suitable for use in the solvent system of the present invention is a polyglycerin. A preferred polyglycerin is a glycerin trimer available under the tradename Polycast-3 from M.M.P., Inc. (South Plainfield, N.J.).

In a particularly preferred embodiment, the alkyl glycol mixture consists of methylpropanediol, butylene diol and pentylene glycol and the polyol ether is a polyglycerin.

Antioxidants suitable for inclusion in the solvent system of the present invention are well-known to persons of skill in the art and are disclosed, for example, in U.S. Pat. No. 6,492,326, the disclosure of which is incorporated herein by reference. Preferred antioxidants that may be used in the solution are selected from the group consisting of butylated hydroxytoluene, propyl gallate, nordihydroguaiaretic acid, ascorbic acid and its derivatives, tocopherol and its derivatives. Preferred Vitamin E derivatives are sold under the tradenames Trolox® and Aquasol E®. A particularly preferred antioxidant according to the present invention is butylated hydroxytoluene, alone or in combination with other antioxidants.

In a preferred embodiment of the present invention, the ratio of the flavonoids) to the solvent system is at least about 1:50, preferably at least about 1:40, more preferably at least about 1:30 and most preferably at least about 1:20.

The CTFA Dictionary describes a wide variety of non-limiting cosmetic and pharmaceutical ingredients that, optionally, are suitable for use in compositions of the present invention. Examples of these ingredient classes include: antimicrobial agents, antioxidants, anti-inflammatory agents, vitamins, trace minerals, film formers and other polymeric materials that increase the substantivity of the compositions of the present invention to the skin, humectants, moisturizers, pH adjusters, skin-conditioning agents, skin soothing and/or healing agents, and sunscreen actives.

The following examples are further illustrative of the present invention. The components and specific ingredients are presented as being typical, and various modifications can be derived in view of the foregoing disclosure within the scope of the invention.

### Examples

Composition

Phytocytol™ BLM is made by first preparing the following solvent system:

| Ingredients | INCI | % (wt/wt) |
|---|---|---|
| Polycast 3 (M.M.P., Inc.) | Polyglycerin | 49.95 |
| Butylene Glycol | Butylene Glycol | 30.00 |
| Soldiol MPD (M.M.P., Inc.) | Methylpropanediol | 10.00 |
| Pentylene Glycol | Pentylene Glycol | 10.00 |
| BHT | BHT | 0.05 |

Separately, three solutions A, B and C, each containing a single flavonoid, are prepared by dissolving the flavonoid into the solvent system:

| | | |
|---|---|---|
| | | Solvent |
| Solution | Flavonoid | System |

| | | | |
|---|---|---|---|
| A | Baicalin MM | 3% | 97% |
| B | Luteolin MM | 6% | 94% |
| C | Myricetin MM | 6% | 94% |

Solutions A, B and C are combined to produce a final Phytocol BLM solution in which baicalin, luteolin and myricetin, respectively, are present at concentrations of 1%, 2% and 2% based on the total weight of the final solution.

Testing

Skin samples from an abdominal plastic surgery patient are placed in a specific explant survival medium BEM from Centre de Recherche Laboratoire BIO-EC (Clamart, France). Once daily, the living skin explants are irradiated with a Vilber Lourmat solar simulator RMX3W (Marne-la-Vallee, France) at a dose of 75 mJ/cm² within a wavelength range of 290-330 nm and a peak of 312 nm (UVB) for 80 seconds.

Five minutes following irradiation, the skin samples are treated with a topical application of 2 mg/cm² of a gel formulation comprising the Phytocytol™ BLM, a stable flavonoid solution according to the present invention. The gel formulation is made according to the following procedure: Three separate gels, each containing one of baicalin, luteolin and myricetin, are prepared according to the same procedure. In a main beaker, add DI Water, Germall Plus and Carbopol Ultrez-10. When Carbopol is dispersed add ¼ of the indicated amount of Neutrol TE. Add Polycast-3 and continue mixing. Add the flavonoid solution (A, B and C) until homogenous. Add the remaining Neutrol TE and mix. The resulting Baicalin Gel, Luteolin Gel and Myricetin Gel are mixed in equal parts to form a gel having a pH of 6.91 and a viscosity of 30,500 cps (LV4@12 rpm).

| | | Baicalin | Luteolin | Myricetin |
|---|---|---|---|---|
| INCI Name | Tradename | Gel | Gel | Gel |
| DI Water | | 30.00 | 30.00 | 30.00 |
| Diazolidinyl Urea | Germall Plus | 0.20 | 0.20 | 0.20 |
| and Iodopropynyl | | | | |
| Butylcarbamate | | | | |
| Carbomer | Carbopol | 0.60 | 0.60 | 0.60 |
| | Ultrez-10 | | | |
| Scutellaria | Baicalin MM | 10.00 | 0.00 | 0.00 |
| Baicalensis | (3% Solution) | | | |
| Root Extract | | | | |
| Citri Reticulatae | Luteolin MM | 0.00 | 10.00 | 0.00 |
| Peel Extract | (6% Solution) | | | |
| Ampelopsis | Myricetin MM | 0.00 | 0.00 | 10.00 |
| Grossedentata | (6% Solution) | | | |
| Extract | | | | |
| Polyglycerin | Polycast-3 | 57.82 | 57.82 | 57.82 |
| Tetrahydroxypropyl | Neutral TE | 1.38 | 1.38 | 1.38 |
| Ethylenediamine | | | | |

Baicalin M M, Luteolin M M, Myricetin M M and Polycast-3 are all commercially available from M.M.P., Inc. (South Plainfield, N.J.). Germall Plus is commercially available from International Specialty Products. Carbopol Ultrez-10 is commercially available from Noveon, Inc. Neutrol TE is commercially available from BASF AG.

A separate group of skin samples are treated with a positive control—a topically-applied gel formulation containing 0.5 percent arbutin, a substance known to those with skill in the art as a whitening agent and inhibitor of tyrosinase. Two sets of control samples are also prepared—the first receives no UV-B irradiation and no treatment (i.e., with a skin-lightener); the second receives UV-B but no skin-lightening treatment.

Culture medium is aspirated at Day 3 and Day 5 and stored at −20° C. On Day 5, the explant samples are divided in two portions. One portion is fixed in formalin, embedded in paraffin and cut into 5 μm sections for histological study. A second portion is frozen at −80° C. and 7 μm cryostat sections are made for subsequent immunofluorescence studies. Respectively, the two portions are used to evaluate inhibition of tyrosinase activity and degree of migration of melanosomes in dendritic appendages, a process referred to hereinbelow as melanocyte dendricity.

Tyrosinase activity in the skin sections is assessed using the method of Laidlaw and Blackberg. (Laidlaw, G F and Blackberg, S. N., Am. J. Pathol. 8:491-498, 1932) Skin tissue samples are incubated in a solution containing 1 mg/ml L-DOPA. Where tyrosinase is active, L-DOPA is oxidized, producing an observable black pigment in the melanocytes. The samples are post-fixed, embedded in paraffin and sectioned. More intense color of the melanocytes indicates high tyrosinase activity and, concomitantly, low skin-lightening activity. Less intense color of the melanocytes indicates inhibition of tyrosinase activity and high-lightening activity.

In non-irradiated, non-treated controls, as well as in non-irradiated samples treated with the composition containing the stable flavonoid solution of the present invention, there is the same high level of tyrosinase activity. This level of tyrosinase activity is substantially greater compared to the arbutin-treated samples. In contrast, in UVB-irradiated samples, the formulation of the present invention shows a substantial reduction in tyrosinase activity compared to the control samples and a lesser reduction in activity compared to the arbutin-treated samples. This indicates that in terms of reduction in tyrosinase activity, the formulation of the present invention is more effective in preventing UVB-induced skin pigmentation than arbutin.

The production of melanin within the melanocytes is observed histologically using a silver-embedding stain according to the Fontana-Masson method, the specifics of which are known to those with skill in the art. In the absence of exposure to UVB irradiation, the non-treated controls, the arbutin-treated controls, and the samples treated with the composition of the present invention, show low levels of melanin deposits. After exposure to UVB irradiation, melanin deposits are observed to moderately increase within the non-treated controls. An increase in melanin deposits is not observed, however, in samples treated with composition of the present invention as well as those treated with arbutin. Thus, the composition of the present invention helps to maintain a comparable level of melanin deposits within the skin independent of UVB irradiation.

Melanocyte dendricity is observed in 7 μm cryostat sections using an immunofluorescent method which employs a mouse monoclonal antibody protein Anti-Melan-A (Monoclonal Clone A103, Santa Cruz Biotechnology, Santa Cruz, Calif. USA) with a biotin-strepavidin amplifying system and fluorescein isothiocyanate (FITC). In non-UVB irradiated samples, a moderate level of melanocyte dendricity is observed in samples treated with the formulation of the present invention. In contrast, in non-treated controls and arbutin-treated samples there is a low level of dendricity. After UVB irradiation, a high level of melanocyte dendricity is seen in the control samples whereas the samples treated with the formulation of the present invention display a comparable moderate level of dendricity (i.e., similar to the non-irradiated state). A low level of dendricity is observed in the arbutin-treated samples similar to the non-irradiated state.

Without wishing to be bound by a theory, applicants believe that skin-lightening compositions comprising the stable flavonoid solution of the present invention reduce UVB-induced formation of melanin by inhibiting tyrosinase activity while not interfering with normal cellular processes of skin pigmentation.

1-20. (canceled)

21. A method of treating a dermatologic condition comprising applying the stable flavonoid solution of claim 1 to the skin.

22. (canceled)

23. The stable flavonoid solution of claim 1 wherein the ratio of the flavonoid or flavonoid derivative to the solvent system is at least about 1:50.

24-26. (canceled)

27. The method of claim 21 wherein the dermatologic condition is selected from the group consisting of inflammation, fine lines and wrinkles associated with biological aging and photoaging, and hair loss.
